(19) United States
(12) Patent Application Publication
(10) Pub. No.: US 2009/0035374 A1
(43) Pub. Date: Feb. 5, 2009

(54) METHOD OF MAKING A SINGLE-STAGE PHARMACEUTICAL PREPARATION FOR ORAL THERAPY TO REGULATE BLOOD PRESSURE AND KIT CONTAINING SAME

(76) Inventors: Katrin Mittmann, Jena (DE); Thomas Zimmermann, Jena (DE)

Correspondence Address: STRIKER, STRIKER & STENBY, 103 East Neck Road, Huntington, NY, 11743, US

(21) Appl. No.: 12/182,220

(22) Filed: Jul. 30, 2008

Related U.S. Application Data
(63) Continuation-in-part of application No. 12/135,470, filed on Jun. 9, 2008.
(60) Provisional application No. 60/952,940, filed on Jul. 31, 2007.

(51) Int. Cl. A61K 9/24; A61P 9/00

(52) U.S. Cl. 424/472

(57) ABSTRACT

The single-stage oral contraceptive preparation contains at least 21 daily dosage units, which each contain a contraceptive combination of 2.0 mg of dienogest (not an aidosterone antognist) and 0.030 mg of ethinyl estradiol, of 2.0 mg of dienogest and 0.020 mg of ethinyl estradiol, of 2.0 mg of dienogest and 0.015 mg of ethinyl estradiol, or of 1.5 mg of dienogest and 0.015 mg of ethinyl estradiol, together with one or more pharmaceutically acceptable excipients and/or carriers, and at the most 7 daily dosage units containing a placebo or no active ingredients. The contraceptive preparation also regulates the blood pressure, raising low blood pressure and lowering mildly elevated blood pressure, during its administration for contraception. It is especially suitable for long-duration use as a contraceptive preparation without risk of negatively influencing the blood pressure.

### CROSS-REFERENCE

This is a continuation-in-part of U.S. patent application Ser. No. 12/135,470, filed on Jun. 9, 2008, which, in turn, contains subject matter that is disclosed in U.S. Provisional Patent Application Ser. No. 60/952,940, filed on Jul. 31, 2007.

### BACKGROUND OF THE INVENTION

1. The Field of the Invention

The subject matter of the present invention includes a method of making a single-stage pharmaceutical preparation for oral therapy to regulate blood pressure, which contains a contraceptive composition of 2.0 mg of 17α-cyano-methyl-17β-hydroxyestra-4,9-dien-3-one (dienogest) and 0.030 mg of 17α-ethinyl estradiol (ethinyl estradiol) or 2.0 mg of dienogest and 0.020 mg of ethinyl estradiol or 2.0 mg dienogest and 0.015 mg ethinyl estradiol or 1.5 mg of dienogest and 0.015 mg of ethinyl estradiol.

2. Description of the Related Art

The significance of cardiovascular disease as a health problem is under-estimated in many cases. For example, 35% of potential patients in the USA consider breast cancer to be their highest health risk and only 7% believe that it is cardiovascular disease.

Hypertension is one of the decisive risk factors for cardiovascular disease. Hypotension, however, should not be disregarded either, because it can have an appreciable effect on the quality of life of the affected individual via its symptoms, such as fatigue, loss of drive, weakness, dizziness, and a tendency to loose consciousness.

Excessively low blood pressure can lead to insufficient blood supply and thus insufficient oxygen supply to the heart, brain and other organs. For pregnant women, low blood pressure is a risk factor, because a causal connection exists between reduced blood pressure, insufficient blood circulation in the uterus, development disorders and perinatal complications.

Naturally, both hypotension and hypertension should be treated with appropriate means primarily by a cardiologist. However the connection between hypertonia/hypotonia and contraception, hormone replacement, and the treatments of gynecological diseases should be considered more intensively by gynecologists.

The blood pressure categories in the following Table I are those given in European Society of Hypertension, 2003—Categorized Blood Pressure Values.

TABLE I
BLOOD PRESSURE VALUE CATEGORIES

| | SYSTOLIC | DIASTOLIC |
|---|---|---|
| CATEGORY | (mmHg) | (MMHg) |
| Optimum Values | <120 | <80 |
| Normal Values | 120-129 | 80-84 |
| High Normal Values | 130-139 | 85-89 |
| Stage 1 Hypertonia (mild) | 140-159 | 90-99 |
| Stage 2 Hypertonia (moderate) | 160-170 | 100-109 |
| Stage 3 Hypertonia (severe) | ≧180 | ≧110 |
| Isolated Systolic Hypertonia | ≧140 | <90 |

For women, hypotonia (low blood pressure)—at first only a measured parameter and not a disorder—is defined by the World Health Organization WHO as a blood pressure of less than 100/60 mg.

It is known that estrogens, especially ethinyl estradiol, activate the renin-angiotensin-aldosterone system (RMS), which regulates blood pressure, by increasing the formation of renin substrates (angiotensinogens). Thus they can assist sodium retention and cause an increase in blood pressure according to W. OELKERS, “Drospirenone: A New Gestagen” in Geburtshilfe und Frauenkunde 61, (2001), pp. 851-856.

Also F. LEIDENBERGER, et al (editors), “Kiinische Endokrinologie für Frauenärzte [Clinical Endocrinology for Gynecologists]”, 3^{rd} complete and expanded ed., publ. by Springer Medizin Verlag, Heidelberg, (2005), p. 192, explains that oral contraceptives containing ethinyl estradiol (EE) act on the rennin-angiotensin system and thus can cause an increase in blood pressure.

R. G. KETELHUT, in “Long-term Discontinuation of Oral Contraceptives”, J. für Hypertonie, 4(2), (2000), pp. 18-21, pointed out that oral contraceptives increase the blood pressure, which is shown by a blood pressure decrease after discontinuation of administration of oral contraceptives.

A. O. MUECK, et al, in “Hormone Therapy and Hypertonia”, J. Hypertonia, 10 (1), (2006), pp. 14-21, likewise explains that oral contraceptives, especially during long-term administration, frequently cause an increase in the blood pressure values, which is usually greater for systolic values than diastolic values. High blood pressure can develop very quickly after administration of an oral contraceptive but blood pressure increases can occur slowly and successively.

W. EIGER, et al, in DE 30 22 337 disclose that oral contraceptives have a negative effect on the blood pressure but the spironolactone compound, dropsirenone, (originally a diuretic of the aldosterone-antagonist type) avoids increasing the blood pressure.

Also W. OELKERS, ibid, have shown that anti-mineralcorticoid-acting gestagens, such as the spironolactone compound, dropirenone, act as aldosterone antagonists and thus lower blood pressure.

### SUMMARY OF THE INVENTION

It is an object of the present invention to provide a suitable agent with contraceptive action and without negative effects on blood pressure.

It is another object of the present invention to provide a kit for contraception that comprises daily dosage units of a pharmaceutical preparation according to the present invention that does not have negative effects on the blood pressure, especially during long-term oral administration.

These objects and others, which will be made more apparent hereinafter, are attained by a method of making a single-stage pharmaceutical preparation for oral therapy to regulate blood pressure, said method comprising preparing at least 21 daily dosage units of the pharmaceutical preparation and including in each of the daily dosage units a contraceptive combination of 2.0 mg of dienogest and 0.030 mg of ethinyl estradiol, or a contraceptive combination of 2.0 mg of dienogest and 0.020 mg of ethinyl estradiol, or a contraceptive combination of 2.0 mg of dienogest and 0.015 mg of ethinyl estradiol, or a contraceptive combination of 1.5 mg of dienogest and 0.015 mg of ethinyl estradiol.

In preferred embodiments of the foregoing method coated tablets (film tablets) are provided for oral administration, each of which consists of a tablet core and a coating extending around the tablet core. Each tablet core contains a part of an entire amount of the dienogest in the combination, which is released in a retarded fashion, and the coating contains another part of the entire amount of the dienogest in the combination, which is released in a non-retarded fashion. Also the entire amount of the ethinyl estradiol in the combination is present in the coating and is released in a non-retarded fashion. Furthermore the coated tablet is prepared so that at least 10%, preferably at least 30%, of the dienogest dissolves from the tablet core in a retarded fashion after more than 30 minutes, as measured by a dissolution test with water at 37° C. as dissolution medium and with a stirring rate of 50 rpm/min. The instructions according to Ph. Eur. call for a paddle mixer as stirring apparatus using 1000 ml of water.

Alternatively the oral dosage unit can be in the form of a tablet with a sugar-containing jacket (dragée).

Also peroral dosage units according to the invention can be: hard gelatin capsules or soft gelatin capsules, which are filled with oil or water suspensions as filling mass or other peroral suspensions.

The foregoing objects are also attained by a kit containing
at least 21 daily dosage units of the aforesaid pharmaceutical composition, each of which contains one of the aforesaid combinations of dienogest and ethinyl estradiol together with one or more pharmaceutically compatible or acceptable auxiliary agents and/or carriers, and
at the most 7 daily dosage units containing no active ingredient or containing a placebo, so that the total number of daily dosage units in the preparation amounts to 28.

In preferred embodiments of the aforesaid kit the number of daily dosage units containing the combination of dienogest and ethinyl estradiol amounts to 21, 22, 23, 24, or 25 and the number of daily dosage units containing no active ingredient or containing a placebo is 7, 6, 5, 4, or 3 so that the kit preferably contains a total number of 28 daily dose units corresponding to the number of days in a 28-day cycle.

According to the present invention one or more of the above-described objects of the present invention are attained by a kit containing
at least n×21 daily dosage units of the aforesaid pharmaceutical composition, each of which contains one of the aforesaid combinations of dienogest and ethinylestradiol, and in which n is 2, 3, 4, 5, 6, 7, 8, 9, 10, 11, 12, 13, 14, 15, 16, or 17, and
at the most 7 daily dosage units containing no active ingredient or containing a placebo.

Preferably the number of daily dose units containing no active ingredient or agent or containing a placebo is 3, 4, 5, 6, or 7.

Depending on the desire of the women for continued control of blood pressure and continued contraception combined with a need for freedom from bleeding over the entire administration time interval the total number of daily dosage units containing the combination of dienogest and ethinyl estradiol in the kit can amount to 84 and the number of daily dosage units containing no active ingredient or containing a placebo can be 7, so that the total number of cycle days per year is 4×(n×21 plus 7), n being equal to 4.

It was unexpected that an oral contraceptive could be formulated that regulates blood pressure (lowers high blood pressure, raises low blood pressure), which contains ethinyl estradiol and dienogest, a gestagen that is not an aldosterone antagonist. This pharmaceutical (contraceptive) combination is thus especially suitable for long-term administration without risk of negative effects.

For women, who desire contraception and suffer from mildly elevated blood pressure and/or whose blood pressure is increased by administration of oral contraceptives, the method of making of the pharmaceutical preparation according to the invention provides a suitable contraceptive agent without the negative effects on their blood pressure. Furthermore for women with high or low blood pressure, who subsequently develop a desire to have children and want to become pregnant, the use of the contraceptive combination of ethinyl estradiol and dienogest according to the present invention reduces the risk of developmental disorders of the fetus and perinatal complications during pregnancy. Furthermore a reduction of the risk of folate-deficiency dependent birth defects during pregnancy, such as neuronal defects; deformities such as split lip, split pallet, split jaw, pregnancy complications, such as premature birth and placenta loss, is possible for these women by addition of METAFOLIN® to the pharmaceutical combination of ethinyl estradiol and dienogest of the present invention.

### EXAMPLES

The following examples illustrate the above-described invention in more detail, but the details in these examples should not be considered as limiting the claims appended herein below.

### Example 1

Valette is a conventional dragée for oral contraception, which contains 0.030 mg of ethinyl estradiol and 2.0 g of dienogest in a tablet core, which is coated by a sugar-containing jacket.

### Example 2

2 mg of dienogest and 0.02 mg of ethinyl estradiol, with 1 mg of dienogest, which is released in a retarded fashion, and 1 mg of dienogest and 0.02 mg of ethinyl estradiol, which are rapidly released.

Description

The example is a coated tablet with a core matrix. The core of the coated tablet contains 1 mg of dienogest in a hydrophilic erosion matrix with METOLOSE® as the basic constituent. The matrix releases the active ingredient, dienogest, in a retarded manner. The core was coated with a rapidly dissolving coating containing 1.0 mg of dienogest and 0.02 mg of ethinyl estradiol. For protection against light, the coated tablet was covered with an additional, rapidly dissolving colored layer containing iron oxide pigments.

The following table 11 describes the composition of the tablet core and tablet coating of example 2.

TABLE II
COMPOSITION OF THE TABLET CORE AND TABLET
COATING OF EXAMPLE 2

| CORE | | COATING FILM | |
|---|---|---|---|
| | | Film 1 - containing | |
| Granulate 1: | | the active ingredient: | |
| Dienogest | 1.000 mg | Dienogest | 1.000 mg |
| METOLOSE ® 90 | 7.500 mg | Ethinyl estradiol | 0.020 mg |
| SH-4000 | | | |
| Lactose monohydrate | 21.000 mg | METHOCEL ® 5 | 2.250 mg |
| Corn starch | 14.000 mg | Talc | 0.0450 mg |
| Povidone K25 | 1.500 mg | Titanium dioxide | 0.280 mg |
| (10% in ethanol) | | | |
| Granulate 2: | | Film 2 - Colored layer: | |
| Lactose monohydrate | 54.000 mg | METHOCEL ® 5 | 3.375 mg |
| Corn starch | 27.100 mg | Talc | 0.675 mg |
| Maltodextrin | 6.900 mg | Titanium dioxide | 1.875 mg |
| (25% in water) | | Iron oxide, red | 0.075 mg |

| Outer phase: | |
|---|---|
| Carboxymethylstarch | 1.500 mg |
| sodium | |
| Magnesium stearate | 1.500 mg |

### Example 3

### Description

The coated tablet, which contains a total dose of 1.5 mg of dienogest and 0.015 mg of ethinyl estradiol, consists of a retarding core matrix and a rapidly dissolving coating, as well as a colored layer.

The following table III describes the composition of the tablet core and tablet coating of example 3.

TABLE III
COMPOSITION OF THE TABLET CORE AND TABLET
COATING OF EXAMPLE 3

| | |
|---|---|
| VARIANT | 8.5 |
| CORE | 104 mg |
| Dienogest | 0.675 mg |
| METOLOSE ® 90SH-4000 | 9.000 mg |
| Lactose monohydrate | 42.925 mg |
| Corn starch | 15.000 mg |
| Povidone K25 (19% in water) | |
| Maltodextrin (25% in water) | 6.000 mg |
| TABLETTOSE ® | 8.500 mg |
| AVICEL ® PH 102 | 7.000 mg |
| Magnesium stearate | 0.900 mg |
| Barrier layer: | |
| EUDRAGIT ® RL 30 D (as coating drier) | |
| Macrogol 6000 | |
| Talc | |
| Active ingredient-containing coating: | |
| Dienogest | 0.825 mg |
| Ethinyl estradiol | 0.015 mg |
| METHOCEL ® 5 | 4.060 mg |
| Talc | 0.836 mg |
| Titanium dioxide | 0.264 mg |
| Colored layer: | |
| METHOCEL ® 5 | 1.500 mg |
| PEG = Macrogol 6000 | 0.300 mg |
| Talc | 0.300 mg |
| Titanium dioxide | 0.850 mg |
| Iron oxide, red | 0.050 mg |

### Example 4

The following table IV describes the composition of the tablet of example IV.

TABLE IV
COMPOSITION OF THE TABLET OF EXAMPLE 4

| CORE | |
|---|---|
| Dienogest | 2.000 mg or 1.500 mg |
| Ethinyl estradiol | 0.015 mg |
| METAFOLIN ® | 0.451 mg |
| Lactose monohydrate | 28.720 mg |
| Corn starch | 15.000 mg |
| Maltodextrin | 3.750 mg |
| Magnesium stearate | 0.500 mg |

An ethinyl estradiol-betacyclodextrin complex can be used in place of ethinyl estradiol. In the event that ethinyl estradiol-beta-cyclodextrin complex (1:2) is used, a maximum or about tenfold amount is to be used.

The substances are mixed in an appropriate fashion and granulated. At the end of the granulation process, METAFOLIN® is applied, the composition is once again mixed, and is then tableted and optionally coated.

### Example 5

The following table V describes the composition of the tablet of example V.

TABLE V
COMPOSITION OF THE TABLET OF EXAMPLE 5

| CORE | |
|---|---|
| Dienogest | 2.000 mg |
| Ethinyl estradiol | 0.030 mg |
| METAFOLIN ® | 0.451 mg |
| Lactose monohydrate | 28.720 mg |
| Corn starch | 15.000 mg |
| Maltodextrin | 3.750 mg |
| Magnesium stearate | 0.500 mg |

An ethinyl estradiol-beta-cyclodextrin complex can be used in place of ethinyl estradiol. In the event that ethinyl estradiol-beta-cyclodextrin complex (1:2) is used, a maximum or about tenfold amount is to be used.

The substances are mixed in an appropriate fashion and granulated. At the end of the granulation process, METAFOLIN® is applied, the composition is once again mixed and is then tabletted and optionally coated.

### Tests of the Effectiveness of the Aforesaid Preparations

### BRIEF DESCRIPTION OF THE DRAWING

The objects, features and advantages of the invention will now be illustrated in more detail with the aid of the following description of the preferred embodiments, with reference to the accompanying figures, in which

FIG. 1 is a graphical illustration of the variations of the average systolic blood pressure in mm of Hg during visits for treatment protocols A and B, and

FIG. 2 is a graphical illustration of the variations of the average diastolic blood pressure in mm of Hg during visits for treatment protocols A and B.

The measurement times are indicated on the X-axes of the blood pressure graphs with S, which represents the time of the screening visit, with B, which means the time of the visit to establish a baseline, and with F, which means the time of the final visit, and with numerical values, which designate the times of the respective visits in weeks, i.e. 8, 12, 25, 38 means 8 weeks, 12 weeks, etc.

It should be noted that the results for the experiments regarding the effectiveness of the aforesaid preparation chiefly pertain to the groups “High Normal” to “Stage 1 (mild) Hypertonia” including the “borderline sub-stage”. Testing for the effectiveness of the contraceptive preparations within the “Stage 2 and Stage 3 Hypertonia” groups was precluded for safety reasons.

Table VI defines the explicit sub-groups for the systolic blood pressure,

TABLE VI
SUB-GROUPS FOR THE SYSTOLIC BLOOD PRESSURE

| Systolic Blood Pressure at | Systolic Blood Pressure at | |
|---|---|---|
| Baseline B in mmHg | Screening S in mmHg | SUB-GROUPS |
| <110 | <110 | Low |
| <110 | 110-129 | Low/normal |
| 110-129 | <110 | Low/normal |
| 110-129 | 110-129 | Normal |
| ≧130 | 110-129 | High/normal |
| 110-129 | ≧130 | High/normal |
| ≧130 | ≧130 | High |
| <110 | ≧130 | Low/high |
| ≧130 | <110 | Low/high |

In a randomized double-blind clinical study 1315 women of ages between 18 and 41 years, who had given their written consent for their participation in the study, were treated according to two different protocols A and B. In protocol A one tablet containing a combination of 2 mg of dienogest and 30 micrograms of ethinyl estradiol was administered each day for 84 days followed by a seven day pause in administration. Then that administration cycle was repeated four times for a total of 364 days.

In the second protocol B, which corresponds to a more conventional administration of a contraceptive combination, one tablet containing a combination of 2 mg of dienogest and 30 micrograms of ethinyl estradiol was administered each day for 21 days followed by a seven day pause for a total of 13 conventional cycles. In other words, protocol B comprised a total of 13 28-day conventional cycles, each comprising 21 days, during which one tablet containing the contraceptive combination of the invention was administered each day, and 7 days, during which no tablet was administered.

Evaluation or analysis of the blood pressure values measured at the initial screening visit, at the baseline visit, in the 10^{th} to 12^{th} treatment weeks, in the 23^{rd} to 25^{th} treatment weeks, in the 36^{th} to 38^{th} treatment weeks and at the final visit (usually after a year of treatment) yielded the following conclusions.

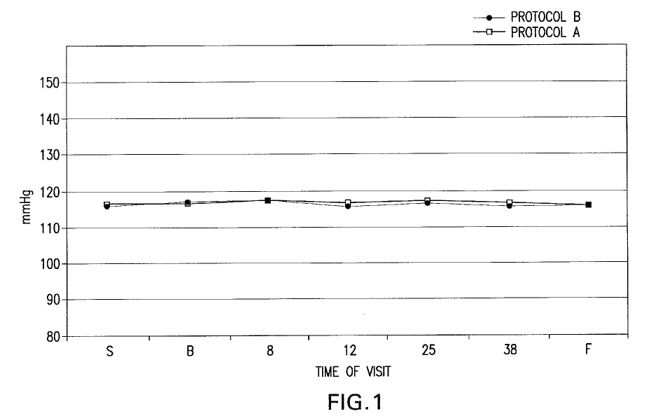

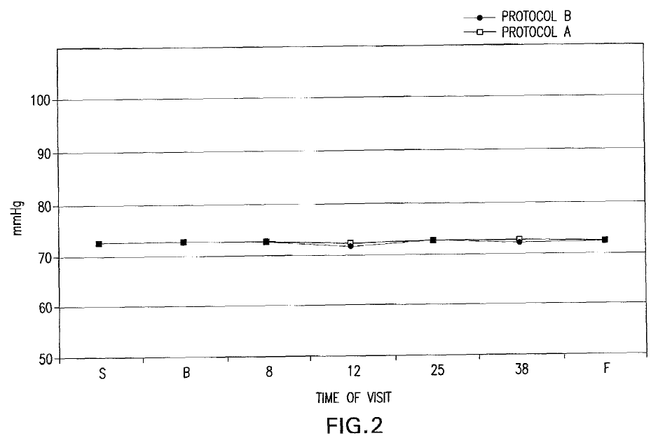

The average systolic blood pressure and the average diastolic blood pressure remains almost constant (see FIGS. 1 and 2) for both group A and group B in the course of the entire study.

If one considers the variation of the average systolic blood pressure in the sub-groups defined in Table VI with respect to the blood pressure values at the baseline and screening visits (see FIG. 2), women in the low/normal sub-group experienced on average a discrete increase of their systolic blood pressure. Women in the high/normal sub-group and/or with slightly elevated blood pressure values experienced on average a discrete decrease in their systolic blood pressure. Women in the normal sub-group were substantially unaffected by administration of the medication. The same results were obtained from the measurements of diastolic blood pressure.

The term “no active ingredient” in the following claims means no ingredient that raises or lowers blood pressure or is effective in preventing conception alone or together with another active ingredient.

While the invention has been illustrated and described as embodied in a method of making a single-stage pharmaceutical preparation for oral therapy to regulate blood pressure and a kit containing the pharmaceutical preparation, the invention is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A method of making a single-stage pharmaceutical preparation for oral therapy to regulate blood pressure, said method comprising preparing at least 21 daily dosage units of the pharmaceutical preparation and including in each of the daily dosage units
a contraceptive combination of 2.0 mg of dienogest and 0.030 mg of ethinyl estradiol, or
a contraceptive combination of 2.0 mg of dienogest and 0.020 mg of ethinyl estradiol, or
a contraceptive combination of 2.0 mg of dienogest and 0.015 mg of ethinyl estradiol, or
a contraceptive combination of 1.5 mg of dienogest and 0.015 mg of ethinyl estradiol.

2. The method as defined in claim 1, further comprising preparing a coated tablet, wherein said coated tablet consists of a tablet core and a coating extending around the tablet core, said tablet core contains a part of an entire amount of said dienogest in said combination, which is released in a retarded fashion, and said coating contains another part of the entire amount of said dienogest in said combination, which is released in a non-retarded fashion, and an entire amount of said ethinyl estradiol, which is released in a non-retarded fashion.

3. The method as defined in claim 2, wherein said coated tablet is prepared so that at least 10% of said dienogest is dissolved out of said tablet core in said retarded fashion after more than 30 minutes, as measured by a dissolution test with water at 37° C. as dissolution medium and with a stirring rate of 50 rpm/min.

4. The method as defined in claim 2, wherein said coated tablet is prepared so that at least 30% of said dienogest is dissolved out of said tablet core in said retarded fashion after more than 30 minutes, as measured by a dissolution test with water at 37° C. as dissolution medium and with a stirring rate of 50 rpm/min.

5. A kit containing a single-stage pharmaceutical preparation for oral therapy to regulate blood pressure, said kit containing
at least 21 daily dosage units of the pharmaceutical preparation, each of said daily dosage units containing
a contraceptive combination of 2.0 mg of dienogest and 0.030 mg of ethinyl estradiol, or
a contraceptive combination of 2.0 mg of dienogest and 0.020 mg of ethinyl estradiol, or
a contraceptive combination of 2.0 mg of dienogest and 0.015 mg of ethinyl estradiol, or
a contraceptive combination of 1.5 mg of dienogest and 0.015 mg of ethinyl estradiol;
together with one or more pharmaceutically acceptable excipients and/or carriers; and
at the most 7 daily dosage units containing no active ingredient or containing a placebo.

6. The kit as defined in claim 5, containing 21, 22, 23, 24, or 25 of said daily dosage units containing the contraceptive combination of the dienogest and the ethinyl estradiol and 7, 6, 5, 4, or 3 of said daily dosage units containing no active ingredient or containing said placebo respectively, so that a total number of said daily dosage units amounts to 28, which corresponds to a 28-day cycle.

7. A kit containing a single-stage pharmaceutical preparation for oral therapy to regulate blood pressure, said kit containing
at least n×21 daily dosage units of a pharmaceutical composition, n being equal to 2, 3, 4, 5, 6, 7, 8, 9, 10, 11, 12, 13, 14, 15, 16, or 17, each of said daily dose units containing
a contraceptive combination of 2.0 mg of dienogest and 0.030 mg of ethinyl estradiol, or
a contraceptive combination of 2.0 mg of dienogest and 0.020 mg of ethinyl estradiol, or
a contraceptive combination of 2.0 mg of dienogest and 0.015 mg of ethinyl estradiol, or
a contraceptive combination of 1.5 mg of dienogest and 0.015 mg of ethinyl estradiol;
together with one or more pharmaceutically acceptable excipients and/or carriers; and
at the most 7 daily dosage units containing no active ingredient or containing a placebo.

8. The kit as defined in claim 7, containing 3, 4, 5, 6, or 7 of said daily dosage units containing no active ingredient or said placebo.

9. The kit as defined in claim 7, containing 84 of said daily dosage units each containing said combination of said dienogest and said ethinyl estradiol, and 7 of said daily dosage units containing no active ingredient or containing said placebo, so that a total number of cycle days per year is 4×(m×21 plus 7), m being equal to 4.
